(19) United States
(12) Patent Application Publication
(10) Pub. No.: US 2026/0306007 A1
(43) Pub. Date: Oct. 8, 2026

(54) SYSTEM FOR INDICATING PROXIMITY TO TRANSEPTAL SITE WITH ELECTRO-ANATOMICAL CATHETER SHEATH

(71) Applicant: Boston Scientific Scimed, Inc., Maple Grove, MN (US)

(72) Inventors: Megan Wurden, Waltham, MA (US); Charles A. Gibson, Malden, MA (US)

(21) Appl. No.: 19/639,919

(22) Filed: Apr. 6, 2026

Related U.S. Application Data
(60) Provisional application No. 63/784,815, filed on Apr. 7, 2025.

(51) Int. Cl. A61B 5/367; A61B 5/00; A61B 5/06; A61B 5/343; A61B 34/20

(52) U.S. Cl. CPC A61B 5/367; A61B 5/062; A61B 5/063; A61B 5/343; A61B 5/746; A61B 34/20; A61B 2034/2046

(57) ABSTRACT

An electroanatomical mapping (EAM) system for use in a cardiac procedure of a heart having a left atrium and an intra-atrial septum including a puncture. The EAM system has a controller, a display, and a catheter sheath having a shaft. The shaft includes tracking electrodes spaced-apart from each other along a longitudinal axis. The catheter sheath is to be inserted through the puncture. The controller determines impedances during the cardiac procedure, in which a current is injected to a current carrying pair of tracking electrodes and voltages are measured via corresponding tracking electrodes. The controller determines a position of each of the tracking electrodes with respect to the left atrium and the intra-atrial septum based on the impedances. The controller also generates, on the display, a graphical representation of the tracking electrodes with respect to the intra-atrial septum based on the determined positions.

### CROSS REFERENCE TO RELATED APPLICATIONS

This application claims priority to U.S. Provisional Patent Application No. 63/784,815 entitled “SYSTEM FOR INDICATING PROXIMITY TO TRANSEPTAL SITE WITH ELECTRO-ANATOMICAL CATHETER SHEATH,” filed Apr. 7, 2025, which is hereby incorporated by reference in its entirety.

### TECHNICAL FIELD

The present disclosure relates to electro-anatomical mapping (EAM) systems and methods for use in cardiac procedures. More specifically, the present disclosure relates to systems and methods for determining and graphically representing the position of a catheter sheath with respect to an intra-atrial septum.

### BACKGROUND

Cardiac procedures requiring access to the left atrium, such as electrophysiology procedures, heart valve interventions, and left atrial appendage treatments, commonly employ transseptal access, in which a catheter sheath is guided through a puncture in the intra-atrial septum from the right atrium into the left atrium. During these procedures, maintaining awareness of the sheath's position relative to the intra-atrial septum is critical. If the distal tip of the sheath retracts from the left atrium back into the right atrium, the clinician must either re-locate the puncture site or re-puncture the septum, interrupting the procedure and increasing patient risk.

Current methods for visualizing sheath position relative to the intra-atrial septum rely on intracardiac echocardiography or fluoroscopy. Fluoroscopy exposes the patient and clinical staff to ionizing radiation, and both modalities require additional imaging equipment or catheters beyond those used for the primary procedure. Further, because the intra-atrial septum is flexible, static geometric shells generated by existing electroanatomical mapping systems do not reliably depict the real-time position of the septum relative to the sheath. A need exists for a system that provides real-time positional awareness of the catheter sheath with respect to the intra-atrial septum without reliance on ionizing radiation or supplemental imaging catheters.

### SUMMARY

In Example 1, an electroanatomical mapping (EAM) system for use in a cardiac procedure of a heart having left atrium and an intra-atrial septum including a puncture therethrough, the EAM system comprising: a catheter sheath having a shaft including a distal section opposite a proximal section and defining a lumen having a longitudinal axis, the distal section having a plurality of tracking electrodes spaced-apart from each other along the longitudinal axis on the distal section of the shaft, the plurality of tracking electrodes including a distalmost tracking electrode, the catheter sheath configured to be inserted through the puncture; a graphical display; and a controller coupled to the catheter sheath, the controller configured to: determine a plurality of impedances during the cardiac procedure, wherein a current is injected to a current carrying pair of tracking electrodes of the plurality of tracking electrodes and a plurality of voltages are measured via corresponding tracking electrodes of the plurality of tracking electrodes; determine a position of each of the plurality of tracking electrodes the with respect to the left atrium and the intra-atrial septum based on the plurality of impedances; and generate, on the graphical display, a graphical representation of the plurality of tracking electrodes with respect to the intra-atrial septum based on the determined positions.

In Example 2, the EAM system of Example 1, wherein the plurality of voltages are measured via corresponding sensing pair of the plurality of tracking electrodes.

In Example 3, the EAM system of Example 2, wherein the impedances are determined from a four-terminal configuration.

In Example 4, the EAM system of any of Examples 2 and 3, wherein the plurality of impedances are determined from multiplexing multiple sets of sensing pairs of tracking electrodes.

In Example 5, the EAM system of any of Examples 1-4, wherein the position of each of the plurality of tracking electrodes is determined via comparing the determined impedance to a threshold value.

In Example 6, the EAM system of Example 5, wherein the position of each of the plurality of tracking electrodes is determined via determining one of the tracking electrodes of the plurality of tracking electrodes is in contact with cardiac tissue.

In Example 7, the EAM system of any of Examples 5-6, wherein determining the position of a tracking electrode of the plurality of tracking electrodes includes determining whether the tracking electrode is in contact with a blood pool.

In Example 8, the EAM system of any of Examples 1-7, wherein the graphical representation includes a presentation of the tracking electrodes in a color from a plurality of colors representative of a number of tracking electrodes determined to be in the left atrium.

In Example 9, the EAM system of any of Examples 1-7, wherein the plurality of tracking electrodes including a distalmost tracking electrode, wherein the controller is further configured to generate an alert if only the distalmost tracking electrode of the plurality of tracking electrodes is disposed in the left atrium.

In Example 10, the EAM system of Example 9, wherein the alert includes a change in color of a graphical representation of the distalmost tracking electrode.

In Example 11, the EAM system of any of Examples 1-10, wherein the controller is further configured to determine a location of the catheter sheath with respect to the heart, and generate, on the graphical display, a graphical representation of the intra-atrial septum with respect to the heart based on the determined position of each of the tracking electrodes with respect to the intra-atrial septum and the determined location of the catheter sheath with respect to the heart.

In Example 12, the EAM system of Example 11, wherein the controller is configured to determine the location of the sheath with respect to the heart via the tracking electrodes.

In Example 13, the EAM system of Example 12, wherein the controller is configured to determine the location of the sheath with respect to the heart via a magnetic tracking assembly on a catheter axially disposed within the lumen.

In Example 14, the EAM system of any of Examples 1-13, wherein the current carrying pair of tracking electrodes include a distalmost tracking electrode of the plurality of tracking electrodes and a proximal-most tracking electrode of the plurality of tracking electrodes.

In Example 15, the EAM system of any of Examples 1-14, wherein the plurality of tracking electrodes is four tracking electrodes.

In Example 16, an electroanatomical mapping (EAM) system for use in a cardiac procedure of a heart having left atrium and an intra-atrial septum including a puncture therethrough, the EAM system comprising: a catheter sheath having a shaft including a distal section opposite a proximal section and defining a lumen having a longitudinal axis, the distal section having a plurality of tracking electrodes spaced-apart from each other along the longitudinal axis on the distal section of the shaft, the catheter sheath configured to be inserted through the puncture; a graphical display; and a controller coupled to the catheter sheath, the controller configured to: determine a plurality of impedances during the cardiac procedure, wherein a current is injected to a current carrying pair of tracking electrodes of the plurality of tracking electrodes and a plurality of voltages are measured via corresponding tracking electrodes of the plurality of tracking electrodes; determine a position of each of the plurality of tracking electrodes the with respect to the left atrium and the intra-atrial septum based on the plurality of impedances; and generate, on the graphical display, a graphical representation of the plurality of tracking electrodes with respect to the intra-atrial septum based on the determined positions.

In Example 17, the EAM system of Example 16, wherein the plurality of voltages are measured via corresponding sensing pair of the plurality of tracking electrodes.

In Example 18, the EAM system of Example 17, wherein the impedances are determined from a four-terminal configuration.

In Example 19, the EAM system of Example 17, wherein the plurality of impedance are determined from multiplexing multiple sets of sensing pairs of tracking electrodes.

In Example 20, the EAM system of Example 16, wherein the position of each of the plurality of tracking electrodes is determined via comparing the determined impedance to a threshold value.

In Example 21, the EAM system of Example 20, wherein the position of each of the plurality of tracking electrodes is determined via determining one of the tracking electrodes of the plurality of tracking electrodes is in contact with cardiac tissue.

In Example 22, the EAM system of Example 20, wherein determining the position of a tracking electrode of the plurality of tracking electrodes includes determining whether the tracking electrode is in contact with a blood pool.

In Example 23, the EAM system of Example 16, wherein the graphical representation includes a presentation of the tracking electrodes in a color from a plurality of colors representative of a number of tracking electrodes determined to be in the left atrium.

In Example 24, the EAM system of Example 16, wherein the plurality of tracking electrodes including a distalmost tracking electrode, the controller is further configured to generate an alert if only the distalmost tracking electrode of the plurality of tracking electrodes is disposed in the left atrium.

In Example 25, the EAM system of Example 24, wherein the alert includes a change in color of a graphical representation of the distalmost tracking electrode.

In Example 26, the EAM system of Example 16, wherein the controller is further configured to determine a location of the catheter sheath with respect to the heart, and generate, on the graphical display, a graphical representation of the intra-atrial septum with respect to the heart based on the determined position of each of the tracking electrodes with respect to the intra-atrial septum and the determined location of the catheter sheath with respect to the heart.

In Example 27, the EAM system of Example 26, wherein the controller is configured to determine the location of the sheath with respect to the heart via the tracking electrodes.

In Example 28, the EAM system of Example 27, wherein the controller is configured to determine the location of the sheath with respect to the heart via a magnetic tracking assembly on a catheter axially disposed within the lumen.

In Example 29, the EAM system of Example 16, wherein the current carrying pair of tracking electrodes include a distalmost tracking electrode of the plurality of tracking electrodes and a proximal-most tracking electrode of the plurality of tracking electrodes.

In Example 30, the EAM system of Example 16, wherein the plurality of tracking electrodes is four tracking electrodes.

In Example 31, an electroanatomical mapping (EAM) system for use in a cardiac procedure of a heart having left atrium and an intra-atrial septum including a puncture therethrough, the EAM system comprising: a catheter sheath having a shaft including a distal section opposite a proximal section and defining a lumen having a longitudinal axis, the distal section having a plurality of tracking electrodes spaced-apart from each other along the longitudinal axis on the distal section of the shaft, the catheter sheath configured to be inserted through the puncture; a graphical display; and a controller coupled to the catheter sheath, the controller configured to: determine a plurality of impedances during the cardiac procedure, wherein a current is injected to a current carrying pair of tracking electrodes of the plurality of tracking electrodes and a plurality of voltages are measured via corresponding tracking electrodes of the plurality of tracking electrodes; determine a position of each of the plurality of tracking electrodes the with respect to the left atrium and the intra-atrial septum based on the plurality of impedances; determine a location of the catheter sheath with respect to the heart; and generate, on the graphical display, a graphical representation of the plurality of tracking electrodes with respect to the intra-atrial septum based on the determined positions and a graphical representation of the intra-atrial septum with respect to the heart based on the determined position of each of the tracking electrodes with respect to the intra-atrial septum and the determined location of the catheter sheath with respect to the heart.

In Example 32, the EAM system of Example 31, wherein the controller is configured to determine the location of the sheath with respect to the heart via the tracking electrodes or via a magnetic tracking assembly on a catheter axially disposed within the lumen.

In Example 33, a method of an electroanatomical mapping (EAM) system for use in a cardiac procedure of a heart having left atrium and an intra-atrial septum including a puncture therethrough, the EAM system coupled to a graphical display and a catheter sheath having a shaft including a distal section opposite a proximal section and defining a lumen having a longitudinal axis, the distal section having a plurality of tracking electrodes spaced-apart from each other along the longitudinal axis on the distal section of the shaft, the catheter sheath configured to be inserted through the puncture, the method comprising: determining a plurality of impedances during the cardiac procedure, wherein a current is injected to a current carrying pair of tracking electrodes of the plurality of tracking electrodes and a plurality of voltages are measured via corresponding tracking electrodes of the plurality of tracking electrodes; determining a position of each of the plurality of tracking electrodes the with respect to the left atrium and the intra-atrial septum based on the plurality of impedances; and generating, on the graphical display, a graphical representation of the plurality of tracking electrodes with respect to the intra-atrial septum based on the determined positions.

In Example 34, the method of Example 33, further comprising presenting, on the graphical display, the tracking electrodes in a color from a plurality of colors representative of a number of tracking electrodes determined to be in the left atrium.

In Example 35, the method of Example 34, further comprising generating an alert if only the distalmost tracking electrode of the plurality of tracking electrodes is disposed in the left atrium wherein the alert includes a change in color of a graphical representation of the distalmost tracking electrode.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system.

FIG. 2 is a schematic diagram of an embodiment of a sheath for use with the electrophysiology system of FIG. 1.

FIG. 3 is a schematic diagram of an embodiment of a controller for use with the electrophysiology system of FIG. 1.

FIG. 4 is a block diagram of an embodiment of a configuration of the controller of FIG. 3.

FIG. 5 is a schematic diagram of an embodiment of a graphical representation generated with the configuration of the controller of FIG. 4.

FIG. 6 is a schematic diagram of an embodiment of another graphical representation generated with the configuration of the controller of FIG. 4.

FIG. 7 is a schematic diagram of an embodiment of still another graphical representation generated with the configuration of the controller of FIG. 4.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

A catheter sheath is often used in cardiac procedures to guide a catheter into a patient’s body and to position the catheter within the heart. For example, a sheath can be used to guide a catheter between heart chambers, such as from the right atrium through the atrial septum to the left atrium, and to facilitate movement of the catheter inside each chamber. Clinicians manipulating a device in the left atrium typically prefer to visualize the sheath and maintain an understanding of the distal tip of the sheath with respect to the atrial septum to prevent the distal tip of the sheath from slipping back into the right atrium. The sheath provides transseptal access, and if the sheath retracts into the right atrium, clinicians spend time trying to find the puncture site or re-puncture the atrial septum. The current practice is to generate a visualization of the sheath with respect to the atrial septum via intracardiac echo catheters or fluoroscopy, which involves the application of ionizing radiation. The atrial septum is relatively flexible so static geometrical shells constructed in cardiac mapping systems are not useful for assessing the position of the sheath with respect to the transseptal puncture site.

This disclosure is directed to systems and methods used to determine the position of catheter elements with respect to cavities or chambers across a septum, such as the intra-atrial septum or left and right atria without the use of ionizing radiation or additional visualization catheters disposed proximate the catheter element. In one embodiment, the systems and methods render a representation of the catheter element with respect to the intra-atrial septum or the left or right atria on a map constructed in real-time. In one embodiment, the systems and methods can alert a clinician if the position of the catheter element is at risk of retracting into the right atrium. In another embodiment, the system and method can determine the location of the intra-atrial septum with respect to other structures of the heart on an anatomical map. The systems and method are described in the context of the clinical setting and catheter ablation for illustration only and is not to be limited to the context of catheter ablation. The disclosure can be applied in other clinical settings and medical procedures as well, including treatments to the heart valve, the left atrial appendage, and all other medical procedures currently known and medical procedures yet to be discovered.

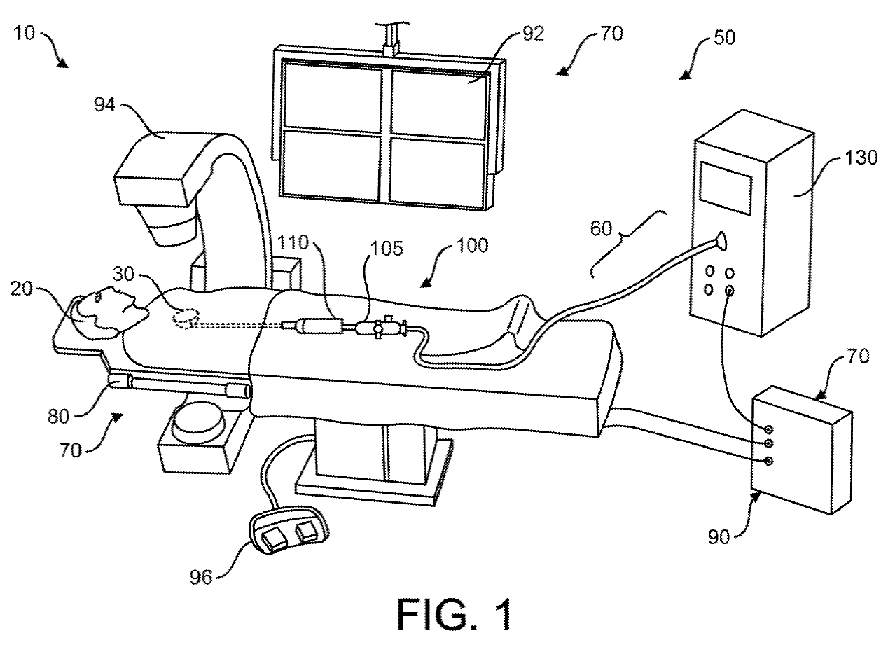

FIG. 1 illustrates an example clinical setting 10 for treating a patient 20, such as for treating a heart 30 of the patient 20, using an electrophysiology system 50, in accordance with the disclosure. The electrophysiology system 50 includes a catheter system 60 and an electroanatomical mapping (EAM) system 70. The example catheter system 60 includes an elongated catheter assembly 100, which includes a plurality of catheter elements. In the illustrated embodiment, the catheter elements of the catheter assembly include a mapping and ablation catheter 105 and a catheter sheath 110. The catheter system 60 also includes an electroporation console 130. Additionally, the catheter system 60 includes various connecting elements, such as cables, that operably connect the components of the catheter system 60 to one another and to the components of the EAM system 70. In general, the EAM system 70 includes a localization field generator 80, a mapping and navigation controller 90, and a display 92. Also, the clinical setting 10 can include additional equipment such as imaging equipment 94 such as a fluoroscopic visualization system (represented by the C-arm) or an echocardiogram system such as an intracardiac echocardiography (ICE) system or a transesophageal electrocardiography (TEE) system (not shown). Additional equipment can also include various controller elements, such as a foot controller 96, configured to allow an operator to control various aspects of the electrophysiology system 50. The clinical setting 10 may have other components and arrangements of components that are not shown in FIG. 1.

An elongated catheter assembly, such as catheter assembly 100, includes a plurality of coaxially disposed catheter elements. For instance, a catheter element defines a longitudinal axis that passes through a centroid of a cross section of the catheter element, such as the centroid of a cross section of a shaft of catheter 105 or a centroid of a cross section of a main lumen of a sheath 110. In the example, the catheter 105 is disposed within the sheath 110. The catheter 105 and sheath 110 are movable with respect to each other along the longitudinal axis. The sheath 110 is operable to provide a delivery conduit through which the catheter 105 can be deployed to the specific target sites within the patient’s heart 30. Access to the patient’s heart can be obtained through a vessel, such as a peripheral artery or vein. Once access to the vessel is obtained, various tools such as the catheter 105 can be navigated to within the patient’s heart, such as within a chamber of the heart.

In the illustrated example of the clinical setting, the example catheter system 60 is configured to deliver ablation energy to targeted tissue in the patient’s heart 30 to create cell death in tissue, for example, rendering the tissue incapable of conducting electrical signals. In some examples, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries. Another ablation technique uses electroporation. In electroporation, or electro-permeabilization, an electrical field is applied to cells to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering.

If the electroporation is irreversible, the affected cells are killed. Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells. The catheter sheath and associated EAM systems and methods described herein are independent of the ablation modality employed and are not limited to use with ablation systems; the systems and methods may be used in any cardiac or other medical procedure requiring transseptal access regardless of whether ablation is performed.

The example catheter 105 includes an elongated catheter shaft and distal end configured to be deployed proximate to the target tissue, such as within a chamber of the patient’s heart. The distal end may include a basket, balloon, spline, configured tip, or other deployment mechanism to effect treatment. In some examples, the deployment mechanism can include an electrode assembly or array having a plurality of electrodes. Each of the plurality of electrodes is electrically coupled to a corresponding elongated lead conductor that extends along the shaft to a catheter proximal end. The lead conductors can be electrically coupled to a plug in the proximal region of the catheter 105, such as a plug configured to be mechanically and electrically coupled to the EAM system 70 and console 130, for example, either directly or via intermediary electrical conductors such as cabling.

The electrode assembly and associated electrodes of a mapping and ablation catheter, such as catheter 105, can be configured for, among other things, sensing cardiac electrical signals, ablation, localization of the electrode assembly within the patient anatomy such as via the EAM system 70, signal reference, and to determine proximity to target tissue within the anatomy. In some embodiments, the catheter 105 is configured for cardiac mapping, and the electrodes are sensing, or mapping, electrodes configured to be used to collect physiological (electrical) signals to be used to generate electroanatomical maps. An example of a physiological signal that the sensing electrode can acquire includes an intracardiac electrogram (ECG) signal. In some embodiments, the catheter 105 can be a mapping and ablation catheter, and the electrodes can include ablation electrodes, or an ablation electrode assembly, that are configured to deliver ablation electric field energy and sensing electrodes, or a sensing electrode assembly, for mapping purposes. In an embodiment of an electroporation catheter, the ablation electrodes in embodiments of an electroporation catheter are configured to receive pulsed electrical signals or waveforms from the console 130 and create pulsed electric fields sufficient to ablate target tissue via irreversible electroporation. The sensing electrodes in the electrode assembly can be electrically coupled to a one or more lead conductors that extends the length of the shaft that are configured to carry an electrical signal received at the sensing electrode. In some examples, an electrode in the electrode assembly can be configured to only perform an ablation or the electrode in the electrode assembly can be configured to only perform mapping. In some examples, an electrode can operate as an ablation electrode in an ablation mode of the electrophysiology system 50 and as a sensing electrode in a sensing or mapping mode of the system 50. Some examples of mapping and ablation catheters are smaller in profile or in the volume of the electrode assembly than catheters that just perform mapping, and clinicians can map a given location within the heart with fewer passes across the chamber with mapping catheters than with mapping and ablation catheters.

The console 130 is configured to control aspects of the catheter system 60. The console 130 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 60. The memory can be part of the one or more controllers, processors, or computers, or part of memory device accessible through a computer network. Examples of computer networks include a local area network, a wide area network, and the internet.

The EAM system 70 is operable to track the location of the various components of the catheter system 60, such as the catheter assembly 100, and to generate high-fidelity three-dimensional anatomical and electro-anatomical maps of the heart, including portions of the heart such as cardiac chambers of interest or other structures of interest such as the sinoatrial node or atrioventricular node. In one illustrative embodiment, the EAM system 70 can include the OPAL™ HDx mapping system marketed by Boston Scientific Corporation. The mapping and navigation controller 90 of the EAM system 70 includes one or more controllers, such as microprocessors or computers, that execute a program or computer readable instructions out of a memory device to control or perform functional aspects of the EAM system 70, in which the memory, can be part of the one or more controllers, microprocessors, computers, or part of a memory device accessible through a computer network.

The EAM system 70 can generate a localization field, via the magnetic field generator 80, to define a localization volume about the heart 30, and a location sensor or sensing element on a tracked device, such as sensors on the electroporation catheter 105, generate an output that can be processed by the mapping and navigation controller 90 to track the location and orientation of the sensor or sensors, and consequently, the corresponding device, within the localization volume. In the illustrated embodiment, the device tracking is accomplished using magnetic tracking techniques, in which the field generator 80 is a magnetic field generator that generates a magnetic field defining the localization volume, and location sensors on the tracked devices are magnetic field sensors.

In other embodiments, impedance tracking methodologies may be employed to track the locations of the various devices. In such examples, the localization field is a set of independently oriented and spatially varying electric fields generated, for example, by an external field generator arrangement, such as surface electrodes, by intra-body or intra-cardiac devices, such as an intracardiac catheter and associated sheath 110, or both. In these examples, the location sensing elements can constitute tracking electrodes on the tracked sheath that generate outputs received and processed by the mapping and navigation controller 90 to track the location of the various location sensing electrodes within the localization volume. For instance, an impedance tracking methodology can employ the use of a patch electrode (not shown) attached to the patient’s body, a current or impedance based can be determined between the tracking electrode on the catheter and the patch electrode and the sheath and the patch electrode.

The EAM system 70 can be equipped for magnetic tracking capabilities, impedance tracking capabilities, or for both magnetic and impedance tracking capabilities. Regardless of the tracking methodology employed, the EAM system 70 utilizes the location information for the various tracked devices, along with cardiac electrical activity acquired by, for example, the electroporation catheter 105 or another catheter, sheath or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

In the case of impedance-based tracking with the EAM system 70, the catheters and sheaths include tracking electrodes disposed on the deflectable portions of the respective shafts. Multiple tracking electrodes can be employed on the deflectable portions of the catheters and sheaths for the EAM system 70 to detect and recreate the curvature of the catheters in the body. In one example, each tracking electrode is coupled to a corresponding lead conductor, or lead wire, which extends along the shaft to the proximal portion where it is coupled to an electrical connector. The electrical connector can be coupled to the EAM system 70 such as via cables.

Sheaths in electrophysiological procedures primary introduce and guide other catheters to within the anatomy. In some examples, sheath 110 is referred to as an EAM sheath having tracking electrodes on a steerable sheath to enable mapping system visualization of the distal portion of the sheath during various therapies and procedures, including without limitation, electrophysiology, left atrial appendage, and heart valve related procedures. Unlike many diagnostic or mapping and ablation catheters used in electrophysiological procedures, sheaths include a large portion of the diameter taken up with a major lumen suitable for receiving diagnostic catheters, ablation catheters and other tools. Accordingly, sheaths often do not have room for magnetic tracking devices and instead incorporate the use of tracking electrodes for impedance tracking. Typical sheaths incorporate minor lumens along the shaft to carry wires that connect to the tracking electrodes. The tracking electrodes are localized using a field map created with a navigation-enabled catheter and surface drivers. If a magnetically tracked catheter is disposed within the sheath, the EAM system 70, in some examples, aligns the longitudinal axis of the sheath with the longitudinal axis of more accurate magnetically detected location of the sheath.

Several constraints are employed in the design and implementation of tracking electrodes and associated lead conductors. Among these constraints include that each tracking electrode and associated lead conductor are to be electrically isolated from one another as well as from other conductive material in the sheath such as a conductive braided member along the length of the shaft. Additionally, precise placement of the tracking electrode on the shaft is desired. For instance, the tracking electrode can be radiopaque or echogenic, and clinicians can use the tracking electrode to visualize placement of the sheath while using fluoroscopy or ultrasound. Also, electrode location with respect to the sheath elements, such as a distal tip, and interelectrode spacing are programmed parameters in several tracking and mapping software programs, and three-dimensional reconstruction and modeling is performed with electrode spacing as a constraint in the modeling curve. Thus, the design and implementation of sheaths using tracking electrodes include lumens with ready access to carry conductor leads.

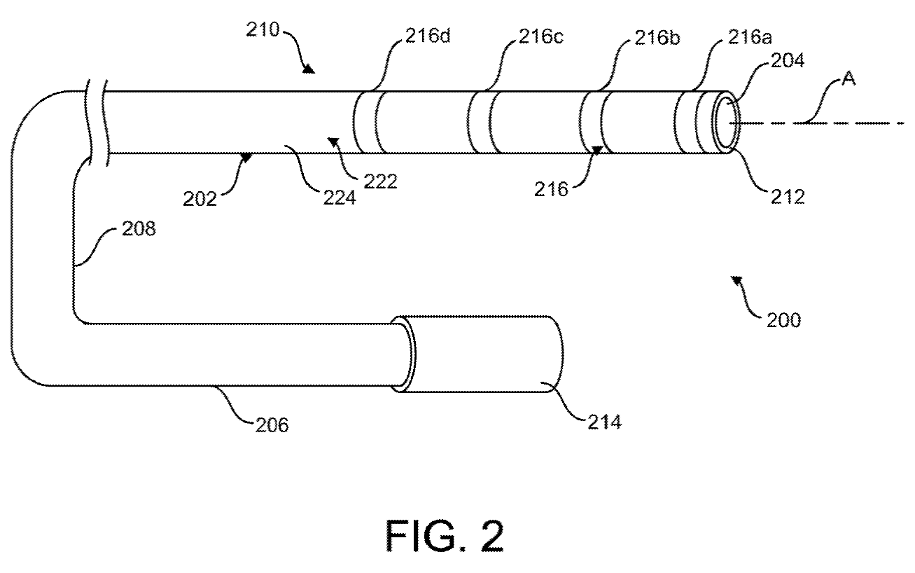

FIG. 2 illustrates an embodiment of a catheter element, such as a sheath 200, that can be used in the example clinical setting 10 in catheter assembly 100. For instance, sheath 200 can be further configured as a guide sheath, dilator, or other flexible, deflectable medical tool that can be tracked via an impedance tracking system such as EAM system 70. In one configuration, the sheath 200 corresponds with catheter sheath 110 of FIG. 1. The sheath 200 includes an elongated shaft 202, such as an elongated and flexible shaft 202 defining a longitudinal axis A. The shaft 202 also defines a major lumen 204 along the longitudinal axis A and has a proximal section 206, an intermediate section 208, and a distal section 210 opposite the shaft 202 from the proximal section 206. The distal section 210 includes a distal tip 212. The proximal section 206 can be coupled to a handle 214 proximal to the shaft 202. In some embodiments, the proximal section 206 or handle 214 can include a hemostatic valve that can be coupled to a source of irrigation fluid and a port to receive catheter elements within the major lumen 204. In some embodiments, portions of the distal section 210 can be configured as a dilator tip.

The sheath 200 includes components disposed along the distal section 210 of the shaft 202. In the illustrated embodiment, the sheath 200 includes a tracking electrode 216, such as a plurality of exposed tracking electrodes 216a…216n, longitudinally spaced apart from each other and longitudinally extending along the distal section 210 of the shaft 202. The illustrated embodiment includes four tracking electrodes 216a, 216b, 216c, 216d, such as a first tracking electrode 216a, a second tracking electrode 216b, a third tracking electrode 216c, and a fourth tracking electrode 216d. In the illustrated embodiment, the first tracking electrode 216a is the distalmost tracking electrode 216a, the second tracking electrode 216b is the second distalmost tracking electrode 216b, the third tracking electrode 216c is the second proximal-most tracking electrode 216c, and the fourth tracking electrode 216d is the proximal-most tracking electrode 216d. In some embodiments, the distalmost tracking electrode is disposed on the distal tip 212. Each tracking electrode 216a…216n is electrically coupled to a respective lead conductor extending along the shaft 202 and terminated at the proximal section 206. In the example, the tracking electrodes 216a…216n are configured for use with an impedance-based tracking system to detect the position of the shaft 202, such as the distal tip 212. In some embodiments, the tracking electrodes 216a…216n are also radiopaque or echogenic. In one embodiment, the plurality of electrodes 216a…216n are formed of an electrically conductive, solid-surface, biocompatible material and are spaced-apart across electrical insulators or electrically insulative portions of the shaft 202. Each electrode 216 of the plurality of electrodes 216a…216n is electrically coupled to an associated elongated lead conductor that extend along the shaft to a catheter proximal end. In one example, each electrode 216 of the spaced-apart electrodes 216a…216n corresponds with a separate, single lead conductor. Other configurations are contemplated. The plurality of lead conductors can be electrically insulated from one another within an electrically insulating covering along the catheter shaft, such as with an electrically insulating polymer cover. The lead conductors can be electrically coupled to plug in the proximal region 206 of the sheath 200, such as a plug configured to be mechanically and electrically coupled to the EAM system 70 or console 130, for example, either directly or via intermediary electrical conductors such as cabling.

In some embodiments, the distal section 210 can include other components disposed on or within the shaft 202, such as pull rings having steering wires disposed along minor lumens in the shaft extending to the proximal section 206 and various other sensors, such as a temperature sensor, a gyroscopic sensor, and an accelerometer within the distal section 210 of the shaft and mechanically and electrically coupled to leads disposed along the shaft and terminated at the proximal section 206. In some embodiments, the shaft 202 includes an irrigation conduit extending from the distal section 210 to the proximal section to supply an irrigation fluid to, for instance, the distal tip 212. The proximal section 206 or the handle 214 in embodiments includes an electrical connection that can be coupled to an impedance-based tracking system, such as EAM system 70. The proximal section 206 or the handle 214 in embodiments includes various controls to operate the steering wires and other connections to other sensors and provide fluid flow.

In some embodiments, the sheath 200 includes a plurality of concentric layers disposed over all of or some of the shaft 202 and along longitudinal axis A, such as a cover member 222 forming an outer surface 224 of the sheath 200, and additional layers underneath the cover member 222 such as a support member that extends along the shaft 202 and are coaxial with the major lumen 204. In various embodiments, the support member is a flexible braid (e.g., stainless steel braid or a high-strength polymer braid, or braid formed from a combination of materials), a coiled shaft, a hypotube (e.g., a laser cut hypotube), or a high-density polyethylene (HDPE) tube. In some embodiments, the shaft 202 also includes a liner layer radially underneath the support member and coaxial with the main lumen 204. In other embodiments, the liner layer is also configured as the support member, and the shaft 202 does not include a braided member or other. The liner layer can define an inner wall of the main lumen 204. In embodiments, the liner layer is a thin wall constructed of polytetrafluoroethylene (PTFE). The support member in some embodiments is a braided member, which can provide characteristics to the sheath 200 such as reduced kinking, wrinkling, or buckling of the shaft 202 and can provide enhanced balance for pushability, deflectability, and torque transmission such as during rotation about the longitudinal axis A. The braided member is constructed from a woven fabric or layer of braided strands or fibers that form interstitial spaces between the fibers. The braided member can be constructed from fibers that include stainless steel fibers, such as electrically conductive fibers, or high strength polymer fibers, or as layers of different materials. In some examples, the support member can include more than one braided member, such as an innermost braided member and an outermost braided member. In some embodiments, the cover member 222 can be formed as a coating of an electrically insulative reflowable plastic or thermoplastic material that extends over the support member and seals underlying components of the shaft 202. For instance, the coating can seep by reflowing over braided material of the support member such as over the fibers and into the interstitial spaces of the braided material of the support member. In one example, the cover member 222 is a polyether block amide and, in some examples, is available under the trade designations PEBAX from Arkema, S.A., and VESTAMID E from Evonik Industries, AG. Electrodes 216 are exposed on the cover member 222. The tracking electrodes 216a…216n are longitudinally spaced-apart from each other on the cover member 222, which also electrically insulates the tracking electrodes 216a…216n from each other.

The depiction of the sheath 200 in FIG. 2 is intended for illustration or general overview of various components of a sheath 200 and is not intended to imply that this disclosure is limited to the number or particular arrangement of tracking electrodes or to the materials and components used to construct the sheath or provide circuit connections. Embodiments of the sheath 200 can include sheaths available under the trade designations FARADRIVE HD, VERSACROSS HD, and SUREFLEX HD from Boston Scientific Corporation.

The electrophysiology system 50 is capable of detecting, or are configured to detect, electrical characteristics, such as impedance, which can correspond with several properties including tissue proximity with respect to a tracking electrode 216 or whether a tracking electrode is in a cardiac chamber on an opposite side of a tissue barrier with respect to another tracking electrode. For example, the system 50 utilizes impedance measurements to sense contact or position with relation to between an electrode 216 on the sheath 200 and cardiac tissue. In another example, the system 50 utilizes impedance measurements of several electrodes to determine an interposition of a medium such as tissue between two or more electrodes.

In general, the impedance of a given medium is determined based upon applying a known voltage or current to a given medium and measuring the resulting current or voltage. In embodiments, the controller, such as controller of the electroporation console 130 or the mapping and navigation controller 90, provides a current between two of the tracking electrodes 216, which are referred to as current carrying pair of electrodes in embodiments, to generate an electric field, and the controller measures voltages at one or more of the electrodes 216, which are referred to as voltage sensing or simply sensing electrodes in embodiments, to determine a responsive voltage to the electric field. In some embodiments, the current is an alternating current. In some embodiments, the alternating current has a constant root-mean-square (RMS) value. In some embodiments, the alternating current has a constant peak-to-peak value. The controller then determines impedance values based on the alternating current provided between the two electrodes 216 and the voltage measurements. Impedance, in some measurements, can include an impedance magnitude and phase angle. In other embodiments, the controller provides a constant direct current between two of the tracking electrodes 216 and the controller measures voltages at one or more of the electrodes. The injected current in some embodiments is a non-stimulating current to reduce the effect on cardiac tissue. In one embodiment, the injected current has a magnitude of 2.5 microamps. The controller then determines impedance values, which are reduced to resistance values, based on the direct current provided between the two electrodes 216 and the voltage measurements. Impedance is determined as the ratio of the measured voltage to the injected current. In one embodiment, the controller compares the impedance values to threshold values to determine how many tracking electrodes are in the left atrium, i.e., the controller determines the location or position of the sheath 200 with respect to the atrial septum.

In some embodiments, impedance measurements can be obtained by injecting a current between two electrodes 216, such as an electrode used a current source and another electrode used as a current sink, and measuring the resulting voltage between one electrode and a reference voltage. The ratio of the voltage potential to the applied current provides an indication of the impedance at the tracking electrode of the voltage measurement. In one embodiment, the controller can inject a current between the distalmost and proximal-most tracking electrodes 216a, 216d, and measure the voltages between tracking electrode 216a and the reference voltage, between tracking electrode 216b and the reference voltage, between tracking electrode 216c and the reference voltage, and between tracking electrode 216d and the reference voltage to obtain the impedance at each tracking electrode 216a…216d in a sheath 200 having four tracking electrodes 216.

In some embodiments, impedance measurements of a given medium is obtained by injecting current between two electrodes and measuring the resulting voltage between the same electrodes through which the current was injected. The ratio of the voltage potential to the applied current provides an indication of the impedance of the medium through which the current traveled, which can be determined via the controller. In one example, a controller, such as controller of the electroporation console 130 or the mapping and navigation controller 90, injects a current between any two electrodes 216 on the sheath 200 and measures a resulting voltage between the same electrodes 216. In this example, impedance is determined in a two-terminal configuration. For example, a current can be injected between the first tracking electrode 216a and the second tracking electrode 216b, and the resulting voltage is measured via the first and second tracking electrodes 216a, 216b. Impedance of the medium (e.g., cardiac tissue) adjacent to first and second tracking electrodes 216a, 216b is measured according to the methodology disclosed above.

In some instances, system 50 may utilize different impedance measurements of a local medium to determine whether the distal ablation electrode 238 is contacting tissue. For example, the impedance of cardiac tissue is different than that of the impedance of blood. Therefore, by knowing the relative difference in the impedances of tissue versus blood, system 50 may be able to determine whether the medium through which a current is being applied is either blood or cardiac tissue, for example.

In some embodiments, impedance is determined in a four-terminal configuration. In general, the four-terminal configuration drives current through a pair of electrodes 216 and measures voltage across a different pair of electrodes 216. For example, current is injected via a current carrying pair of electrodes and voltage is measured across a sensing pair of electrodes. In one advantageous application, a four-terminal configuration may not be sensitive to the impedance of the electrodes themselves. For instance, the measured impedance in a two-terminal configuration includes the surrounding medium and both electrodes. In contrast, the four-terminal configuration measures the voltage across the sensing pair of electrodes in which the current is negligible. Consequently, the measured impedance is that of the surrounding medium and is largely independent of the impedance of the sensing pair of electrodes and their interface with the surrounding medium.

In one example, a current is injected between the distalmost tracking electrode 216a and the proximal-most tracking electrode 216d, which define the current carrying pair of electrodes. The second distalmost tracking electrode 216b and the second proximal-most tracking electrode 216c can define the sensing electrode pair. The current is injected between the distalmost and proximal-most tracking electrodes 216a, 216d, and voltage is measured across the second distalmost and second proximal-most tracking electrodes 216b, 216c. The impedance measurement is the impedance of the medium between the sensing pair of electrodes, or the medium adjacent the second distalmost and second proximal-most tracking electrodes 216b, 216c in this example. In another example, a current can be injected between the distalmost tracking electrode 216a and the second distalmost tracking electrode 216b, which define the current carrying pair of electrodes. The second proximal-most tracking electrode 216c and the proximal-most tracking electrode 216d can define the sensing electrode pair, and voltage is measured across the second proximal-most and proximal-most tracking electrodes 216c, 216d. The impedance measurement is the impedance of the medium between the sensing pair of electrodes, or the medium adjacent the second proximal-most and proximal-most tracking electrodes 216c, 216d in this example.

In some embodiments, the four-terminal configuration is implemented via three electrodes, such as one of the electrodes (a dual-purpose electrode in the configuration) is included in the current carrying pair of electrodes and the sensing pair of electrodes. This configuration can approximate the four-terminal configuration if the impedance of the dual-purpose electrode is low and not expected to vary significantly. For example, a current can be injected between the distalmost tracking electrode 216a and the proximal-most tracking electrode 216d, which define the current carrying pair of electrodes. The second distalmost tracking electrode 216b and the distalmost tracking electrode 216a can define the sensing electrode pair, and voltage is measured across the second distalmost and distalmost tracking electrodes 216b, 216a. The impedance measurement is the impedance of the medium between the sensing pair of electrodes, or the medium adjacent the second distalmost and distalmost tracking electrodes 216b, 216a. Additionally, one or more of the electrodes in a four-terminal configuration can be located off the sheath 200, such as on a pad coupled to the patient’s skin or on another surgical device. In some embodiments, any pair of tracking electrodes 216 can implemented as current carrying pair of electrodes from which current is injected, and any pair of tracking electrodes 216 can be implemented as the sensing pair of electrodes across which voltage is measured and consequently impedance is determined.

Impedance measurements are applied to assess whether or which of the tracking electrodes have passed through the atrial septum into the left atrium or back into the right atrium and then determine the distal tip 212 of the sheath from those measurements. In one embodiment, the current is injected between the distalmost tracking electrode 216a and the proximal-most tracking electrode 216n, or in the illustrated embodiment, 216d. Other current carrying pairs of tracking electrodes are contemplated. Voltage is measured from various sensing electrodes 216 such as via cycling through the tracking electrodes or multiple sensing pairs of electrodes using multiplexed drivers. As the set of tracking electrodes 216 is moved with respect to the atrial septum, such as through a puncture site in the atrial septum, the septal tissue moves with relation to the current carrying pair of electrodes, and the voltages at the sensing electrodes change. The impedance at a sensing electrode is different relative to the other tracking electrodes when the atrial tissue is in contact with the electrode. The impedance of a sensing pair of electrodes is different when the electrodes are on either side of the atrial septum (the sensing pair of electrodes are disposed in opposite cardiac chambers) than a sensing pair of electrodes disposed in the same cardiac chamber. Impedance measurements are periodically taken to determine changes in the position of the sheath with respect to the atrial septum and are tracked to determine which of the tracking electrodes are disposed in which chamber.

Based upon the determined locations of the tracking electrodes 216 with respect to the atrial septum, the controller generates a visualization including an indication of whether the distal tip 212 of the sheath 200 is proximate the atrial tissue in the left atrium. For instance, if only the distalmost tracking electrode 216a is in the left atrium and the remaining tracking electrodes are in the right atrium as determined by the impedance measurements, the controller generates an alert such as an audio signal or a visual indicator, such as to display a representation of the catheter on a visualization of the heart in an electroanatomic map with respect to the heart and illustrate the distalmost tracking electrode as an warning the user, such as in a color such as red or as a flashing light. In one embodiment, if two tracking electrodes 216a, 216b are in the left atrium as determined by the impedance measurements, the controller generates an indication such as to display the representation of the sheath 200 on the visualization of the heart in electroanatomical map with the distalmost two electrodes 216a, 216b represented in yellow. In one embodiment, if three tracking electrodes 216a, 216b, 216c are in the left atrium as determined by the impedance measurements, the controller generates an indication such as to display the representation of the sheath 200 on the visualization of the heart with the distalmost three electrodes 216a, 216b represented in orange, and if four or more tracking electrodes 216 are in the left atrium as determined by the impedance measurements, the controller generates an indication such as to display the representation of the sheath 200 on the visualization of the heart with the electrodes 216 in the left atrium in green.

In some embodiments, the location of the atrial septum with respect to the tracking electrodes 216 as determined from the impedance measurements is used to illustrate a representation of the atrial septum in the visualization of the heart on the electroanatomical map. The location of the sheath 200 with respect the heart is determined based on tracking techniques, and the location of the atrial septum with respect to the tracking electrodes is determined via impedance measurements. The controller then determines the location of the atrial septum with respect to the heart and can incorporate the determined location of the atrial septum into the geometry of maps on the heart to inform the septal sections of both the left and right atria.

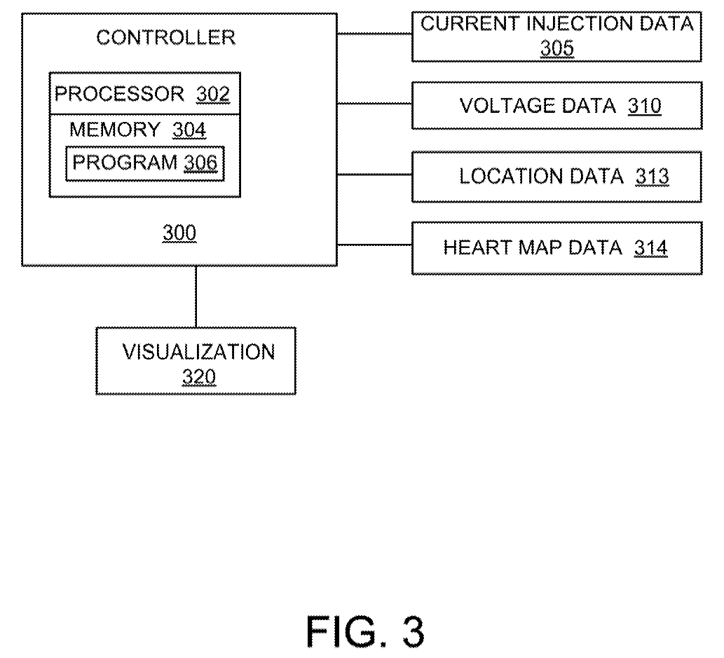

FIG. 3 illustrates an example controller 300 that can be used with the example electrophysiology system 50, such as a controller of the example electroporation catheter system 60, which may include a controller of the electroporation console 130, or a controller of the example EAM system 70, which may include a mapping and navigation controller 90. The controller 300 is configured to be coupled to a catheter sheath, such as sheath 110 and sheath 200, and electrically coupled to the tracking electrodes 216a…216n spaced apart, electrically insulated from each other, and extending along the longitudinal axis. The controller 300 can include a processor 302 and a memory 304. The memory 304 stores processor executable instructions 306. In one example, the processor executable instructions 306 can be in the form of a program, such as a computer program or application. In one example, the controller 300 can be implemented to include a computing device such as a laptop computer, a workstation, a desktop computer, or a tablet. The controller 300 can be implemented to include or be coupled to additional components such as a graphical display, a touchscreen, speakers or other output devices, a keyboard or other input devices, or communication circuitry such as computer network adapters. In one example, the controller 300 is coupled to display 92. The controller 300 may be implemented in a variety of architectures and components, such as the processor 302 and memory 304, may be distributed in various locations. In some embodiments, the processor 302 includes one or more main processing cores to run an operating system and perform tasks on an integrated circuit, and the processor 302 can also include built-in logic or a programmable functional unit, also on the same integrated circuit. Memory 304 is an example of computer storage media. Computer storage media includes RAM, ROM, EEPROM, flash memory or other memory technology, CD-ROM, digital versatile discs (DVD) or other optical storage, magnetic cassettes, magnetic tape, magnetic disk storage or other magnetic storage devices, USB flash drive, flash memory card, or other flash storage devices, or other storage medium that can be used to store the desired information and that can be accessed by the processor 302. Any such computer storage media may be part of the controller 300 and implemented as memory 304. Memory 304 is a non-transitory, processor readable memory device. Accordingly, a propagating signal by itself does not qualify as storage media or memory 304.

The controller 300 is configured to receive inputs from and generate outputs to the electrophysiology system 50. For example, the controller 300 can generate outputs such as current injection data 308, to the console 130, to generate an injection current in tracking electrodes 216 of the sheath 200, or to generate the injection current directly to the sheath 200 for use in generating an electric field to measure impedances. Also, the controller 300 can receive voltage data 310 representative of measured voltages at the tracking electrodes 216. Additionally, the controller 300 can receive location data 312 such as information regarding the location of the sheath with respect to the heart from the EAM system 70. The controller 300 receives an input representative of the anatomical map of the heart, or heart map data 314, which heart map data 314 can include data regarding representations of the geometric anatomical map of the heart and the electro-anatomical map of the heart, such as from the EAM system 70. Further, the controller 300 generates a visualization 320, such as transmits to a graphic display a signal for a visualization of a representation of the sheath with respect to representations of the heart.

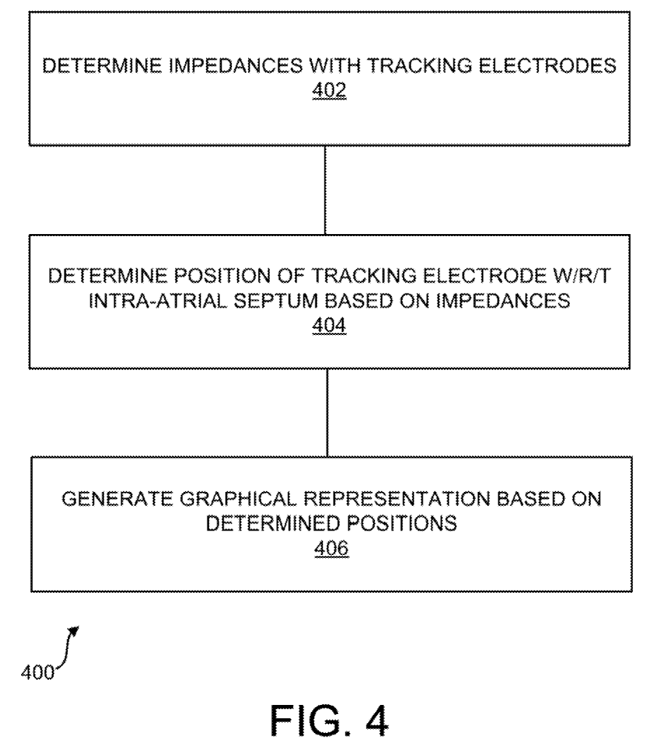

FIG. 4 illustrates a process 400 to configure a controller, such as controller 300. In one embodiment, process 400 can be implemented as set of processor-executable instructions, such as instructions 306, stored in a non-transitory memory, such as memory 304 to be executed by a processor 302 to configure controller 300. The instructions to implement process 400 can be configured to receive information, such as to retrieve from memory 304, voltage data 310 regarding measured voltages, location data 312, and heart map data 314. Further, the instructions to implement process 400 can be configured to perform impedance measurements, annotate, adjust, or write to heart map data 314 and to generate a visualization, such as visualization 320 to transmit to a display such as display 92. The graphical representation can include a schematic representation or other indicia that presents an amount of length of the distal section 210 of the sheath located in the left atrium and distal to the intra-atrial septum such as a number of the tracking electrodes located in the left atrium as may be determined from the current injection data 308 and the voltage data 310. Also, the graphical representation can include a representation that presents the intra-atrial septum with respect to the representation of the heart as may be determined from the current injection data 308, the voltage data 310, and location data 312. In one embodiment, the process is implemented quickly to generate renderings of the locations of the catheter elements with respect to the intra-atrial septum with little lag, such as within one millisecond.

Impedances are determined during the cardiac procedure in which a current is injected to a current carrying pair of tracking electrodes and voltages are measured via corresponding tracking electrodes at 402. Impedances are determined via the current injection data 308 and voltage data 310. The voltage data 310 is determined from measurement circuitry coupled to the catheter sheath and is in response to the injected current. The current is injected between two tracking electrodes, such as the distalmost tracking electrode and the proximal-most tracking electrode, which in the illustrated electrode sheath having four tracking electrodes correspond with tracking electrode 216a and tracking electrode 216d. A set of voltages are measured for the injected current. In some embodiments, the measured voltages include voltages between each tracking electrode and a reference voltage. In some embodiments, the measured voltages include voltages between sensing pairs of tracking electrodes, such as between the distalmost tracking electrode 216a and the second tracking electrode 216b. In one embodiment, the voltages are measured between each pair of tracking electrodes from the possible combinations of tracking electrodes as pairs of sensing electrodes for the injected currents. In some embodiments, voltages, and thus impedances, are determined from multiplexing multiple sets of sensing pairs of tracking electrodes. In some embodiments impedances are determined from multiple injected current using different current carrying pairs of tracking electrodes for each set of voltage measurements. Impedances are determined as a ratio of voltage from voltage data 310 to the injected current from current injection data 308. The controller 300 determines the impedances from values stored in memory 304.

A position of each of the tracking electrodes the with respect to the left atrium and the intra-atrial septum are determined based on the measured impedances at 404. Several mechanisms can be used to determine the location of the tracking electrodes with respect to the left atrium and the intra-atrial septum, and combinations of mechanisms can be applied for a more robust determination of the position of the tracking electrodes. In some embodiments, the impedance at each of the tracking electrodes can be applied to determine the position of each of the tracking electrodes. For example, a measured impedance can be indicative of whether the electrode is in contact with cardiac tissue or in a blood pool. A determination of which medium the tracking electrode is in contact with can be made by comparing the impedance to a threshold value. If the measured impedance of one tracking electrode indicates the tracking electrode is in contact with cardiac tissue and the measured impedance of an adjacent tracking electrode or adjacent tracking electrodes indicate contact with a blood pool, an inference can be made that the tracking electrode is at the intra-atrial septum and the other electrodes are disposed in the left or right atria based on their distal or proximal locations relative to the one electrode. In another embodiment of a mechanism, the movement of the sheath is tracked as it is traversed across the intra-atrial septum and a series of tracking electrodes are measured with impedance indicative of contact with cardiac tissue. For example, the position of the tracking electrodes can be updated as the distalmost tracking electrode 216a corresponds with a measured impedance of contact with cardiac tissue, then the second distalmost tracking electrode 216b corresponds with a measured impedance of contact with cardiac tissue, and then the second proximal-most tracking electrode 216c corresponds with a measured impedance of contact with cardiac tissue. An inference can be made that the distalmost and second distalmost tracking electrodes 216a, 216b are in the left atrium and the proximal-most tracking electrode 216d is in the right atrium.

In another embodiment, the position of each of the tracking electrodes the with respect to the left atrium and the intra-atrial septum are determined based on the measured impedances in combination with the location tracking mechanism of the EAM system 70. In one example, if the measured impedance of one tracking electrode, such as the proximal-most tracking electrode 216d, indicates the tracking electrode is in the blood pool and a recent impedance measurement indicates that the tracking electrode was in contact with cardiac tissue, the measured impedances of the other tracking electrodes also indicate contact with a blood pool, an initial inference can be made that proximal-most electrode 216d in this illustration is in either the left or right atrium and the remaining tracking electrodes are in the left atrium. If the locating tracking information further indicates the sheath was moved further into the left atrium since the recent impedance measurement indicated that the proximal-most tracking electrode 216d was in contact with cardiac tissue, an inference can be made that all of the tracking electrodes 216 are in the left atrium and sheath 200 intersects the intra-atrial septum proximal to the proximal-most tracking electrode 216d in this illustration.

In some embodiments, the impedance of the medium between tracking electrodes is applied to determine the position of each of the tracking electrodes. For example, the impedance measured between two tracking electrodes spanning the intra-atrial septum will be different than an impedance measured between two tracking electrodes, such as adjacent tracking electrodes, disposed in either atrium of the intra-atrial septum for a given injection current. In one embodiment of a mechanism, a current is injected to a current carrying pair of tracking electrodes, such as the distalmost and proximal-most tracking electrodes 216a, 216d, and impedances are determined in the media between the distalmost tracking electrode and the second distal most tracking electrode, between the second distal tracking electrode and the second proximal tracking electrode, and between the second proximal-most tracking electrode and the proximal-most tracking electrode, for three impedance measurements. In this example, the voltages are measured between adjacent tracking electrodes to determine the impedance of the medium between the adjacent tracking electrodes, and impedance can be determined between non-adjacent tracking electrodes in other embodiments. If, an impedance measured is indicative of the tracking electrodes spanning the intra-atrial septum, as compared to the other two measured impedances, an inference can be made that the sheath intersects the intra-atrial septum between the two tracking electrodes having the measured impedance indicative of spanning the intra-atrial septum such as the distal tracking electrode of the two tracking electrodes is in the left atrium and the proximal tracking electrode of the two tracking electrodes is in the right atrium. If the two tracking electrodes are adjacent electrodes, the position of the sheath with respect to the intra-atrial septum is determined with more precision and that an inference can be made that the intra-atrial septum intersects the sheath between the two adjacent tracking electrodes.

In some embodiments, the impedance measurements are enhanced with additional information to increase sensitivity and specificity of the location of the intra-atrial septum in relation to the sheath 200. In some instances, impedance measurements may not have a strong enough characteristic change to determine intra-atrial septum movement through electrode pairs. Other information used in connection with impedance measurements includes respiratory motion of the patient, electrograms (EGMs) received from the tracking electrodes or sensing electrodes on another catheter element including near and far field designations of the EGMs, and differential voltages of the tracking electrodes.

Once the position of the intra-atrial septum is determined with reference to a tracking electrode or a pair of tracking electrodes, the position of the remaining tracking electrodes are determined with respect to the intra-atrial septum and the left and right atria. Any tracking electrodes distal on the sheath to the determined position of the intra-atrial septum with respect to a tracking electrode in contact with the intra-atrial septum are in the left atrium, and any tracking electrodes proximal on the sheath to the determined position of the intra-atrial septum with respect to a tracking electrode in contact with the intra-atrial septum are in the right atrium. Also, any tracking electrodes distal on the sheath to the determined position of the intra-atrial septum with respect to a distal tracking electrode in an adjacent pair of tracking electrodes spanning the intra-atrial septum are in the left atrium, and any tracking electrodes proximal on the sheath to the determined position of the intra-atrial septum with respect to a proximal tracking electrode in an adjacent pair of tracking electrodes spanning the intra-atrial septum are in the right atrium.

A graphical representation of the of tracking electrodes with respect to the intra-atrial septum is generated based on the determined positions of the tracking electrodes with respect to the intra-atrial septum at 406. The graphical representation includes a visualization including a representation of a tracking electrode on representation the distal portion of the sheath with respect to a representation of the cardiac tissue generated via an anatomical map system such as EAM system 70. The EAM system 70 generates a graphical representation of the various tracked devices including a tracked electrode on the tracked sheath within the geometric anatomical map or the electro-anatomical map. In one embodiment, a plurality of the tracking electrodes are represented on the visualization. For instance, all of the tracking electrodes are represented on the visualization along with the positions of each tracking electrode with respect to the intra-atrial septum are represented on the visualization. In another embodiment, a tracking electrode is highlighted in the visualization.

Clinicians often retract the sheaths while manipulating catheter elements, and the distal tip of the sheath can be retracted close to the intra-atrial septum in the left atrium. If the distal tip of the sheath is close to the intra-atrial septum, the clinician may prefer to advance the distal tip further into the left atrium to lessen the risk of the distal tip being retracted into the right atrium. The graphical representation of the of tracking electrodes with respect to the intra-atrial septum is generated based on the determined positions of the tracking electrodes with respect to the intra-atrial septum to avoid unintended retractions of the distal tip of the sheath into the right atrium and to allow the clinician to determine whether the distal tip of the sheath is safely within the left atrium.

In one embodiment, the graphical representation includes a presentation of the tracking electrodes in a color from a plurality of colors representative of a number of tracking electrodes determined to be in the left atrium. For instance, if three or more tracking electrodes are determined to be located in the left atrium, the representation of the tracking electrodes in the left atrium can be highlighted in a color such as green. This can provide an indication to the EAM system operator that the sheath is well within the left atrium. If, however, only two tracking electrodes are determined to be located in the left atrium, the representation of the tracking electrodes in the left atrium can be highlighted in a color such as yellow or orange. This can provide an indication to the EAM system operator that the sheath is close to the intra-atrial septum, and further movement of either the septum, the sheath, or both, could cause the sheath to be retracted back to the right atrium. If only one tracking electrode is determined to be located in the left atrium, the representation of the tracking electrode in the left atrium can be highlighted in a color such as red. This can provide an indication to the EAM system operator that the sheath is close to the intra-atrial septum, and further movement of either the septum, the sheath, or both, could cause the sheath to be retracted back to the right atrium.

In some embodiments, the controller is further configured to generate an alert if only the distalmost tracking electrode of the plurality of tracking electrodes is disposed in the left atrium. In some embodiments, only the distalmost tracking electrode is determined to be located in the left atrium, the distalmost tracking electrode in the left atrium can be highlighted in a flashing color such as a flashing red light as an alert. This can provide an indication to the EAM system operator that the sheath is very close to the intra-atrial septum, and further movement of either the septum, the sheath, or both, could cause the sheath to be retracted back to the right atrium. In another embodiment, the alert can be accompanied by an audio signal provided to another output device, such as speakers coupled to the controller. In one embodiment, the alert is disabled if the distal tip 212 of the catheter is in a continuous movement from the right atrium into the left atrium.

In some embodiments, a graphical representation of the intra-atrial septum with respect to the heart is generated based on the determined position of each of the tracking electrodes with respect to the intra-atrial septum and the location of the catheter sheath with respect to the heart at 408. In some examples of EAM systems, the intra-atrial septum is flexible and static geometric shells used in mapping systems for rendering of cardiac tissue struggle to accurately depict the puncture site or the location of the septum. Using the determined position of the tracking electrodes with respect to the intra-atrial septum and the determined location and curvature of the sheath from the EAM system, the EAM system can generate a representation of the location of the intra-atrial septum with respect to the anatomical map. The incorporation of detailed, real-time representations of the intra-atrial septum with respect to the geometry of the heart in anatomical maps can be used to inform the septal portions of the right and left atria. In some embodiments, the incorporation of the representation of the septum into the anatomical map can be automatic or performed manually via editing tools. In one embodiment, the sheath is localized via impedance tracking. In other embodiments, the tracking electrodes are localized using a field map created with a navigation-enabled catheter and surface drivers. If a magnetically tracked catheter is disposed within the sheath, the EAM system 70, in some examples, aligns the longitudinal axis of the sheath with the longitudinal axis of more accurate magnetically detected location of the sheath. The sheath is localized via a combination of impedance tracking and magnetic tracking.

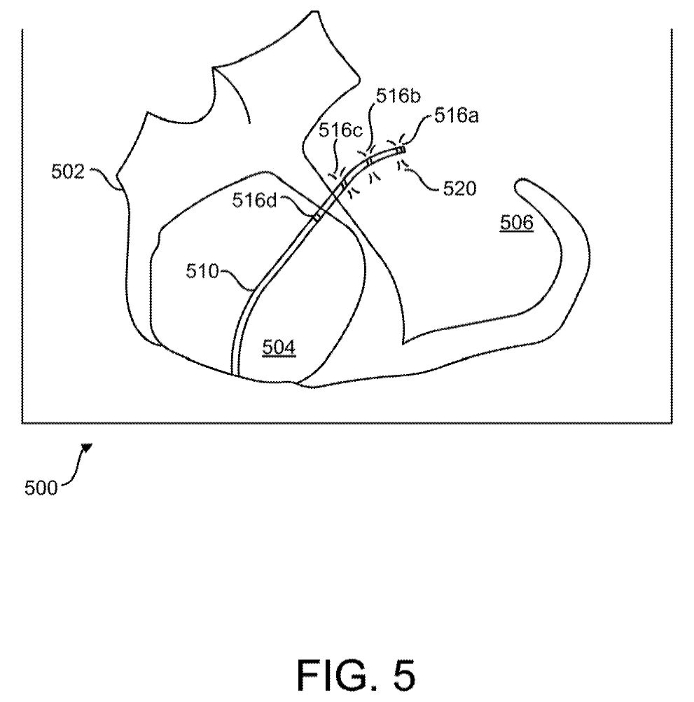

FIG. 5 is an example graphical representation 500 of tracking electrodes with respect to the intra-atrial septum generated based on the determined positions of the tracking electrodes with respect to the intra-atrial septum and facilitated as a visualization in a display as configured by processor 300 using process 400. The graphical representation includes an anatomical map of the heart 502 illustrating the right atrium 504 and left atrium 506. The representation of the sheath 510 includes representations of the tracking electrodes 516, such as the representation of the distalmost tracking electrode 516a, the second distalmost tracking electrode 516b, the second proximal most tracking electrode 516c, and proximal most tracking electrode 516d associated with the positions of the actual sheath 200 and corresponding tracking electrodes 216 with respect to the cardiac tissue, such as determined according to the EAM system 70. To determine the positions of the tracking electrodes with respect to the intra-atrial septum, the impedances are determined in which a current is injected to a current carrying pair of tracking electrodes and voltages are measured via corresponding tracking electrodes. For instance, a current is injected between the distalmost tracking electrode 216a and the proximal-most tracking electrode 216d, which corresponds with the distalmost tracking electrode 516a and the proximal-most tracking electrode 516d of the graphical representation. Impedances are determined from measuring voltages between adjacent tracking electrodes, such as between the distalmost tracking electrode 216a and second distalmost tracking electrode 216b, between the second distalmost tracking electrode 216b and the second proximal-most tracking electrode 216c, and between the second proximal-most tracking electrode 216c and the proximal-most tracking electrode 216d. In this example, the voltages are measured between adjacent tracking electrodes to determine the impedance of the medium between the adjacent tracking electrodes. The measured impedances can be compared to each other or to threshold values to determine a position of the intra-atrial septum with respect to the tracking electrodes. Once the location of the intra-atrial septum is determined with respect to tracking electrodes, the positions of the remaining electrodes with respect to the atria are determined in this example. In the illustrated example, the proximal-most tracking electrode 216d and second proximal-most tracking electrode 216c are determined to be the adjacent tracking electrodes spanning the intra-atrial septum. The proximal-most tracking electrode is determined to be in the right atrium and the remaining tracking electrodes 216a-216c, all distal to the proximal-most electrode 216c on the sheath 200, are determined to be in the left atrium. The graphical representation 500 includes the representation of the of tracking electrodes 516a-516c in the left atrium 506 and the representation of the proximal-most tracking electrode 516d in the right atrium 504. The positioning of the distal tip 212 of the sheath is relatively safe from being imminently retracted across the septum, and thus no alerts are provided on the graphical representation 500. In one example, the graphical representation 500 can render the representations of the three tracking electrodes in the left atrium as green (indicated by light rays 520 near tracking electrodes 516a, 516b, 516c).

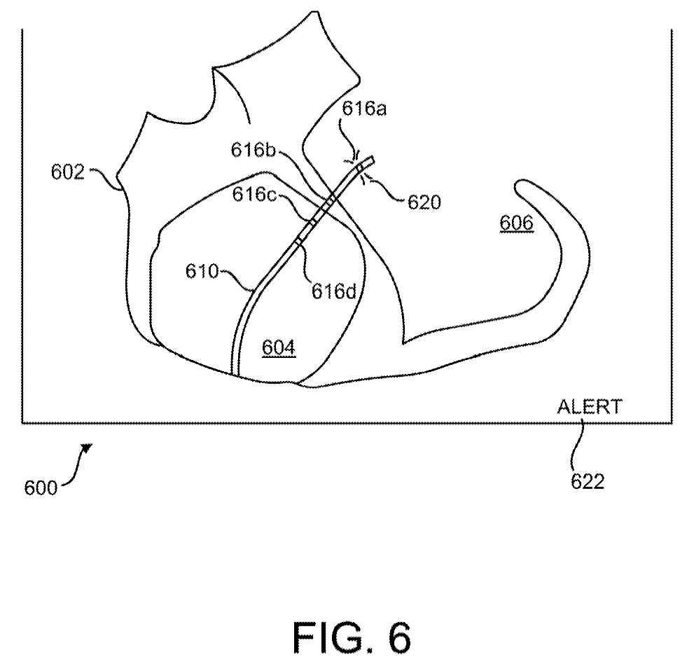

FIG. 6 is an example graphical representation 600 of tracking electrodes with respect to the intra-atrial septum generated based on the determined positions of the tracking electrodes with respect to the intra-atrial septum and facilitated as a visualization in a display as configured by processor 300 using process 400. The graphical representation 600 includes an anatomical map of the heart 602 illustrating the right atrium 604 and left atrium 606. The representation of the sheath 610 includes representations of the tracking electrodes 616, such as the representation of the distalmost tracking electrode 616a, the second distalmost tracking electrode 616b, the second proximal most tracking electrode 616c, and proximal most tracking electrode 616d associated with the positions of the actual sheath 200 and corresponding tracking electrodes 216 with respect to the cardiac tissue, such as determined according to the EAM system 70. The impedances are determined as in the example of FIG. 5, and the position of the intra-atrial septum is also determined as in the example of FIG. 5. In the example of FIG. 6, however, the distalmost tracking electrode 216a and second distalmost tracking electrode 216b are determined to be the adjacent tracking electrodes spanning the intra-atrial septum. The distalmost tracking electrode 216a is determined to be in the left atrium and the remaining tracking electrodes 216b-216d, all proximal to the distalmost electrode 216a on the sheath 200, are determined to be in the right atrium. The graphical representation 600 includes the representation of the of tracking electrodes 616b-616d in the right atrium 604 and the representation of the distalmost tracking electrode 616a in the left atrium 606. The positioning of the distal tip 212 of the sheath risks being imminently retracted across the septum, and thus an alert is provided on the graphical representation such as a flashing red light 620, a notice on the representation 622, or an audio signal.

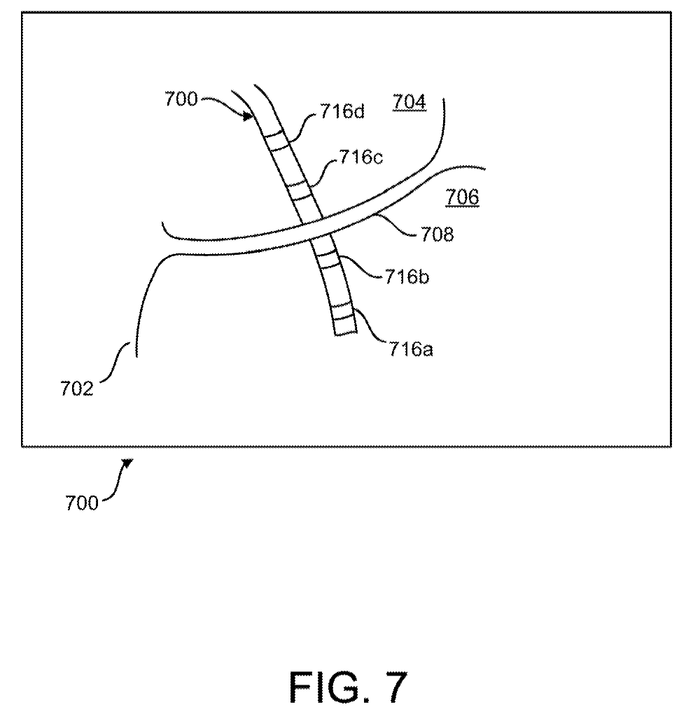

FIG. 7 is an example graphical representation 700 of tracking electrodes with respect to the intra-atrial septum generated based on the determined positions of the tracking electrodes with respect to the intra-atrial septum and facilitated as a visualization in a display as configured by processor 300 using process 400. The graphical representation 700 includes an anatomical map of the heart 702 including the right atrium 704, left atrium 706, and intra-atrial septum 708. The representation of the sheath 710 includes representations of the tracking electrodes 716, such as the representation of the distalmost tracking electrode 716a, the second distalmost tracking electrode 716b, the second proximal most tracking electrode 716c, and proximal most tracking electrode 716d associated with the positions of the actual sheath 200 and corresponding tracking electrodes 216 with respect to the cardiac tissue, such as determined according to the EAM system 70. Using process 400 to determine the position of the intra-atrial septum with respect to the tracking electrodes 216, a determination is made that the second distalmost tracking electrode 216b and second proximal-most tracking electrode 216c are the adjacent tracking electrodes spanning the intra-atrial septum. Further, using location information tracking the location of the sheath with respect to the cardiac anatomy, such as impedance tracking, the intra-atrial septum and its position with respect to the cardiac anatomy and with respect to the representations of the tracking electrodes 716 is rendered on the graphical representation 700. The graphical representation illustrates a representation of the right and left atria 704, 706 and intra-atrial septum 708, and their positions with respect to the renderings of the map of the heart 702 and sheath 710.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An electroanatomical mapping (EAM) system for use in a cardiac procedure of a heart having left atrium and an intra-atrial septum including a puncture therethrough, the EAM system comprising:
a catheter sheath having a shaft including a distal section opposite a proximal section and defining a lumen having a longitudinal axis, the distal section having a plurality of tracking electrodes spaced-apart from each other along the longitudinal axis on the distal section of the shaft, the catheter sheath configured to be inserted through the puncture;
a graphical display; and
a controller coupled to the catheter sheath, the controller configured to:
determine a plurality of impedances during the cardiac procedure, wherein a current is injected to a current carrying pair of tracking electrodes of the plurality of tracking electrodes and a plurality of voltages are measured via corresponding tracking electrodes of the plurality of tracking electrodes;
determine a position of each of the plurality of tracking electrodes the with respect to the left atrium and the intra-atrial septum based on the plurality of impedances; and
generate, on the graphical display, a graphical representation of the plurality of tracking electrodes with respect to the intra-atrial septum based on the determined positions.

2. The EAM system of claim 1, wherein the plurality of voltages are measured via corresponding sensing pair of the plurality of tracking electrodes.

3. The EAM system of claim 2, wherein the impedances are determined from a four-terminal configuration.

4. The EAM system of claim 2, wherein the plurality of impedances are determined from multiplexing multiple sets of sensing pairs of tracking electrodes.

5. The EAM system of claim 1, wherein the position of each of the plurality of tracking electrodes is determined via comparing the determined impedance to a threshold value.

6. The EAM system of claim 5, wherein the position of each of the plurality of tracking electrodes is determined via determining one of the tracking electrodes of the plurality of tracking electrodes is in contact with cardiac tissue.

7. The EAM system of claim 5, wherein determining the position of a tracking electrode of the plurality of tracking electrodes includes determining whether the tracking electrode is in contact with a blood pool.

8. The EAM system of claim 1, wherein the graphical representation includes a presentation of the tracking electrodes in a color from a plurality of colors representative of a number of tracking electrodes determined to be in the left atrium.

9. The EAM system of claim 1, wherein the plurality of tracking electrodes including a distalmost tracking electrode, and the controller is further configured to generate an alert if only a distalmost tracking electrode of the plurality of tracking electrodes is disposed in the left atrium.

10. The EAM system of claim 9, wherein the alert includes a change in color of a graphical representation of the distalmost tracking electrode.

11. The EAM system of claim 1, wherein the controller is further configured to determine a location of the catheter sheath with respect to the heart, and generate, on the graphical display, a graphical representation of the intra-atrial septum with respect to the heart based on the determined position of each of the tracking electrodes with respect to the intra-atrial septum and the determined location of the catheter sheath with respect to the heart.

12. The EAM system of claim 11, wherein the controller is configured to determine the location of the sheath with respect to the heart via the plurality of tracking electrodes.

13. The EAM system of claim 12, wherein the controller is configured to determine the location of the sheath with respect to the heart via a magnetic tracking assembly on a catheter axially disposed within the lumen.

14. The EAM system of claim 1, wherein the current carrying pair of tracking electrodes include a distalmost tracking electrode of the plurality of tracking electrodes and a proximal-most tracking electrode of the plurality of tracking electrodes.

15. The EAM system of claim 1, wherein the plurality of tracking electrodes is four tracking electrodes.

16. An electroanatomical mapping (EAM) system for use in a cardiac procedure of a heart having left atrium and an intra-atrial septum including a puncture therethrough, the EAM system comprising:
a catheter sheath having a shaft including a distal section opposite a proximal section and defining a lumen having a longitudinal axis, the distal section having a plurality of tracking electrodes spaced-apart from each other along the longitudinal axis on the distal section of the shaft, the catheter sheath configured to be inserted through the puncture;
a graphical display; and
a controller coupled to the catheter sheath, the controller configured to:
determine a plurality of impedances during the cardiac procedure, wherein a current is injected to a current carrying pair of tracking electrodes of the plurality of tracking electrodes and a plurality of voltages are measured via corresponding tracking electrodes of the plurality of tracking electrodes;
determine a position of each of the plurality of tracking electrodes the with respect to the left atrium and the intra-atrial septum based on the plurality of impedances;
determine a location of the catheter sheath with respect to the heart; and
generate, on the graphical display, a graphical representation of the plurality of tracking electrodes with respect to the intra-atrial septum based on the determined positions and a graphical representation of the intra-atrial septum with respect to the heart based on the determined position of each of the tracking electrodes with respect to the intra-atrial septum and the determined location of the catheter sheath with respect to the heart.

17. The EAM system of claim 16, wherein the controller is configured to determine the location of the sheath with respect to the heart via the plurality of tracking electrodes or via a magnetic tracking assembly on a catheter axially disposed within the lumen.

18. A method of an electroanatomical mapping (EAM) system for use in a cardiac procedure of a heart having left atrium and an intra-atrial septum including a puncture therethrough, the EAM system coupled to a graphical display and a catheter sheath having a shaft including a distal section opposite a proximal section and defining a lumen having a longitudinal axis, the distal section having a plurality of tracking electrodes spaced-apart from each other along the longitudinal axis on the distal section of the shaft, the catheter sheath configured to be inserted through the puncture, the method comprising:
determining a plurality of impedances during the cardiac procedure, wherein a current is injected to a current carrying pair of tracking electrodes of the plurality of tracking electrodes and a plurality of voltages are measured via corresponding tracking electrodes of the plurality of tracking electrodes;
determining a position of each of the plurality of tracking electrodes the with respect to the left atrium and the intra-atrial septum based on the plurality of impedances; and
generating, on the graphical display, a graphical representation of the plurality of tracking electrodes with respect to the intra-atrial septum based on the determined positions.

19. The method of claim 18, further comprising presenting, on the graphical display, the tracking electrodes in a color from a plurality of colors representative of a number of tracking electrodes determined to be in the left atrium.

20. The method of claim 19, further comprising generating an alert if only the distalmost tracking electrode of the plurality of tracking electrodes is disposed in the left atrium wherein the alert includes a change in color of a graphical representation of the distalmost tracking electrode.
